(19) United States
(12) Patent Application Publication
(10) Pub. No.: US 2017/0304452 A1
(43) Pub. Date: Oct. 26, 2017

(54) EMULSION COMPOSITION, POWDERY MATTER AND PRODUCTION METHOD OF EMULSION COMPOSITION

(71) Applicants: Ryo Teruya, Naha-shi (JP); Mohd Razip Bin Asaruddin, Kajang (MY); Zulfikri Hakim Bin Mohd Razip, Kajang (MY)

(72) Inventor: Mohd Razip Bin Asaruddin, Kajang (MY)

(73) Assignees: Ryo Teruya, Naha-shi (JP); Mohd Razip Bin Asaruddin, Kajang (MY); Zulfikri Hakim Bin Mohd Razip, Kajang (MY)

(21) Appl. No.: 15/497,777

(22) Filed: Apr. 26, 2017

(30) Foreign Application Priority Data
Apr. 26, 2016 (JP) 2016-087886

(51) Int. Cl. A61K 47/24; A23L 2/52; A23L 29/10; A61K 9/107; A61K 47/10; A61K 36/889

(52) U.S. Cl. CPC A61K 47/24; A61K 47/10; A61K 36/889; A23L 29/10; A61K 9/107; A23L 2/52; A23V 2002/00

(57) ABSTRACT

An objet of the present invention is to provide an emulsion composition, a powdery matter and a production method of an emulsion composition, each enabling superior stability of the emulsion.

[Means for Resolution]

An emulsion composition according to one embodiment of the present invention contains a coconut oil, a lecithin and a co-emulsifier. The co-emulsifier is preferably a polyhydric alcohol having at least two hydroxyl groups. The co-emulsifier is preferably propylene glycol, glycerol, sorbitol, xylitol, maltitol, mannitol, glucose, galactose, mannose, lactose, fructose, maltose, sucrose, xylose, trehalose or guar gum. The emulsion composition preferably further contains deionized water. The emulsion composition preferably further contains honey, collagen, inositol, glutathione or potassium sorbate. A powdery matter according to another aspect of the present invention contains a coconut oil, a lecithin and a co-emulsifier. The powdery matter is preferably water soluble.

### FIELD OF INVENTION

The present invention relates to an emulsion composition, a powdery matter and a production method of an emulsion composition.

### DISCUSSION OF THE BACKGROUND

Coconut oils, especially virgin coconut oils (hereinafter, may be also referred to as “VCO”) are edible oils that have been consumed in tropical countries for several thousands of years. VCO contains saturated fatty acids such as lauric acid, myristic acid, palmitic acid and caprylic acid. Since VCO is not heated in the production step, beneficial substances such as e.g., vitamins, anti-oxidants, minerals, medium chain fatty acids and proteins are included therein without disappearance. In addition, the shelf life of VCOs is longer than those of other edible oils, accompanied by less possible decomposition. Accordingly, VCO is advantageous in many aspects.

Moreover, previous investigations have revealed that VCO may be effective in inhibiting Parkinson's syndrome, Alzheimer's disease, dementia and the like. VCO has been reported to be capable of increasing a basal metabolic rate, and to give health benefits to patients suffering from hypothyroidism. VCO is capable of reducing appetite and increasing fat burning, and further can inhibit microbial infections. In addition, VCO is capable of providing athletes with benefits of sustaining durable energy levels for a longer time period without ingestion of drugs or stimulants. Furthermore, VCO raises the good cholesterol HDL (High Density Lipoproteins) level and lower the bad cholesterol LDL (Low Density Lipoproteins) level, whereby arterial occlusion can be prevented, which may lead to prophylaxis of heart diseases.

Although VCO is beneficial in various aspects as described above, some drawbacks have been also found. Examples of such drawbacks include the taste and smell of coconuts. All people do not necessarily like the taste and smell of coconuts, and some people would not be capable of withstanding the unique taste and smell. In addition, when VCO is applied onto the skin, the unique smell of VCO may remain on the skin, and/or clogging of sweat glands may occur. Due to its oily nature, it is difficult to incorporate a water soluble substance into the VCO. Since pure VCOs contain lauric acid which is in a pro-active form, it would be impossible to achieve maximal therapeutic effects by transdermal absorption. Lauric acid has to be activated by converting it into monolaurin which is in an active form of lauric acid in order to achieve satisfactory therapeutic effects.

To solve such problems, incorporation of a glycerin fatty acid ester into foods and beverages containing coconut milk was proposed (see Japanese Unexamined Patent Application. Publication No. 2003-325147). The coconut milk can be emulsified owing to the glycerin fatty acid ester used therein having a hydrophilic group and a hydrophobic group; however, the emulsion stability attained by such emulsification is not yet sufficient.

### PRIOR ART DOCUMENTS

### Patent Documents

Patent Document 1: Japanese Unexamined Patent Application, Publication No. 2003-325147

### SUMMARY OF THE INVENTION

The present invention was made in view of the foregoing circumstances, and an object of the invention is to provide an emulsion composition, a powdery matter and a production method of an emulsion composition, each enabling superior emulsion stability.

### Means for Solving the Problems

According to an aspect of the present invention made for solving the aforementioned problems, an emulsion composition contains a coconut oil, a lecithin, and a co-emulsifier.

Since the emulsion composition contains a lecithin and a co-emulsifier, the emulsion stability can be improved.

The co-emulsifier is preferably a polyhydric alcohol having at least two hydroxyl groups. When the co-emulsifier is the polyhydric alcohol having at least two hydroxyl groups, the emulsion stability can be further improved.

The polyhydric alcohol is preferably glycerol. When the polyhydric alcohol is glycerol, the emulsion stability can be further improved.

It is preferred that as the co-emulsifier, glucose, galactose, mannose, lactose, fructose, maltose, sucrose, xylose, trehalose, propylene glycol, sorbitol, xylitol, mannitol, erythritol, threitol, arabitol, dulcitol, iditol, ribitol, lactitol, maltitol or isomalt is further contained. When the compound is further contained as the co-emulsifier, the emulsion stability can be further improved.

It is preferred that the emulsion composition further contains deionized water. When the emulsion composition contains further deionized water, fermentation of the emulsion composition is enabled.

It is preferred that the emulsion composition further contains honey, collagen, inositol, glutathione or potassium sorbate. When the emulsion composition contains honey, collagen, inositol, glutathione or potassium sorbate, the emulsion composition can be superior in emulsion stability, as well as in functional properties.

A powdery matter according to another aspect of the present invention made for solving the aforementioned problems contains a coconut oil, a lecithin, and a co-emulsifier. Due to being in the form of a powder, the powdery matter is superior in handleability. By dissolving the powdery matter in an aqueous medium, an emulsion composition that is superior in emulsion stability can be obtained.

The powdery matter is preferably water soluble. When the powdery matter is thus water soluble, easy ingestion of the powdery matter can into the body through dissolving water or the like is enabled.

A production method of an emulsion composition according to still another aspect of the present invention made for solving the aforementioned problems includes a mixing step of mixing a coconut oil, a lecithin, and a co-emulsifier.

Due to including the mixing step of mixing a coconut oil, a lecithin and a co-emulsifier, an emulsion composition that is superior in emulsion stability can be produced by the production method of the emulsion composition.

### Effects of the Invention

The emulsion composition and the powdery matter according to the aspects of the present invention are superior in stability of the emulsion due to containing the lecithin and the co-emulsifier. Furthermore, according to the production method of an emulsion composition of the still another aspect of the present invention, an emulsion composition that is superior in emulsion stability can be produced.

### BRIEF DESCRIPTION OF THE DRAWINGS

FIG. 1 shows an SEM image illustrating the emulsion composition of Example 1.

FIG. 2 shows an SEM image illustrating the emulsion composition of Example 2.

### DESCRIPTION OF EMBODIMENTS

Hereinafter, preferred modes for carrying out the invention will be described in detail.

### First Embodiment

### <Emulsion Composition>

The emulsion composition contains a coconut oil, a lecithin, and a co-emulsifier. Since the emulsion composition contains the lecithin, the coconut oil is emulsified by the lecithin, whereby the stability of the emulsion can be improved. Moreover, since the emulsion composition contains the co-emulsifier, a micellar structure, an inverse micellar structure, a vesicular structure, a lamellar structure and the like obtained by the emulsification is strengthened by the co-emulsifier, and thus the emulsion stability can be further improved. In addition, it is preferred that the emulsion composition further contains an enhancer such as honey, collagen, inositol, glutathione or potassium sorbate. Each component of the emulsion composition will be described in detail below.

(Coconut Oil)

The coconut oil is a component that constitutes the emulsion composition, and as described above, the coconut oil has been reported to be effective not only in inhibiting, for example, Parkinson's syndrome, Alzheimer's disease, dementia and the like, but also for hypothyroidism. In addition, the coconut oil is capable of reducing appetite and increasing fat burning, and further can inhibit microbial infections. Also, the coconut oil enables durable energy levels to be sustained for a longer time period, and further raises the good cholesterol HDL (High Density Lipoproteins) level and lower the bad cholesterol LDL (Low Density Lipoproteins) level, whereby arterial occlusion can be prevented, which may lead to prophylaxis of heart diseases.

The coconut oil is preferably a virgin coconut oil. The virgin coconut oil is obtained by pressing coconut meat at a low temperature, without subjecting to bleaching, purification and the like. When the virgin coconut oil is selected as the coconut oil, an emulsion composition good for the health can be obtained.

(Lecithin)

Since the lecithin has a hydrophilic moiety and a hydrophobic moiety (i.e., lipophilic moiety), adding the lecithin to the coconut oil enables the coconut oil to be emulsified, the emulsion stability of the emulsion composition can be improved. In addition, by emulsifying the coconut oil with the lecithin, the coconut oil is confined by the lecithin, whereby the unique taste and smell of the coconut oil can be controlled.

The lower limit of the average diameter of the micellar structures in the state in which the coconut oil is emulsified by the lecithin is preferably 0.01 μm, more preferably 0.05 μm, and even more preferably 0.1 μm. On the other hand, the upper limit of the average diameter is preferably 1.0 μm, more preferably 0.8 μm, and even more preferably 0.6 μm. When the average diameter falls within the above range, the emulsion stability of the emulsion composition can be improved. The “average diameter” as referred to means a median diameter (particle diameter equivalent to 50% of the cumulative distribution) obtained by a laser diffraction particle size distribution analyzer (“LA-750”, manufactured by HORIBA, Ltd.).

(Co-Emulsifier)

The emulsion composition contains the co-emulsifier. Due to containing the co-emulsifier in the emulsion composition, the emulsion can be stabilized.

The co-emulsifier is exemplified by polyhydric alcohols having at least two hydroxyl groups, and the like. It is believed that the hydroxyl groups enhance bonding among the lecithin, the coconut oil, a solvent, the enhancer (to be explained herein later) and the like, and thus the emulsion can be stabilized.

Examples of the polyhydric alcohol include glycerol, glucose, galactose, mannose, lactose, fructose, maltose, sucrose, xylose, trehalose, propylene glycol, sorbitol, xylitol, mannitol, erythritol, threitol, arabitol, dulcitol, iditol, ribitol, lactitol, maltitol, isomalt and the like, which may be used either alone or in combination of two or more thereof. Of these, the polyhydric alcohol is preferably glycerol. When the polyhydric alcohol is glycerol, the stabilization of the emulsion can be further improved.

Furthermore, it is more preferred that as the co-emulsifier, the polyhydric alcohol other than glycerol is used in addition to glycerol. Accordingly, emulsion stabilization can be further improved.

It is to be noted that as the co-emulsifier, in addition to the polyhydric alcohol or in place of the polyhydric alcohol, for example, Konjac gum, gelatin, gum arabic, guar gum, tragacanth, xanthan gum, karaya gum, tara gum, gellan gum, agar, carrageenan, locust bean gum, arabinogalactan, eucheuma seaweed, alginic acid, methyl cellulose, etc., may be also used.

The lower limit of the concentration of the co-emulsifier is preferably 5% by mass, more preferably 8% by mass, and even more preferably 10% by mass. On the other hand, the upper limit of the concentration of the co-emulsifier is preferably 20% by mass, more preferably 18% by mass, and even more preferably 15% by mass. When the concentration of the co-emulsifier falls within the above range, the stability of the emulsion can be further improved.

(Enhancer)

It is preferred that the emulsion composition further contains the enhancer. The enhancer is exemplified by honey, collagen, inositol, glutathione, potassium sorbate and the like. These may be used either alone or in combination of two or more thereof. These can enhance various types of functions of the emulsion composition as described below.

### [Honey]

Honey is a substance that is used as a binder, a thickening agent and a natural additive, and can stabilize the emulsification of the emulsion composition. Honey enables the fats to be dispersed in an emulsion, and thus the emulsion structures can be miniaturized.

### [Collagen]

Collagen is an important protein that serves in connecting cells each other in the living body. Collagen is constituted from amino acids that are essential for human body. The collagen extracted from fishes (marine collagen) has been recognized to be safe, and considered to prevent a human body from infectious diseases. Due to having a low content of hydroxyproline and a low denaturation temperature, marine collagen is superior in digestion and absorption as compared with other collagen.

Collagen is suitably used in the production of acidic foods and beverages. VCO is acidic, and collagen can stabilize the emulsion in the emulsion composition containing such VCO. Due to a strong binding force, collagen enables the emulsion structures to be miniaturized and a stable time period of the emulsion to be prolonged, and further decomposition of the emulsion composition can be inhibited for a long period of time.

### [Inositol]

Inositol is a substance that is indispensable for cell membranes, and constitutes a part of phospholipids in the cell membranes. Furthermore, inositol is involved in the metabolism of cholesterol, fats and calcium. Along with acetylcholine, inositol is necessary for the formation of the lecithin in the human body. In addition, inositol is a substance indispensable for the hair growth, and reportedly, those who are taking a larger amount of inositol exhibit a prolonged time until the hair loss, accompanied by less fallen hair. Moreover, inositol is believed to: lower the cholesterol level; and further to relax nervous tension, make the sleep better, and alleviate severe anxiety, panic attacks and melancholia. Additionally, inositol is believed to prevent rashes. Furthermore, inositol is capable of miniaturizing the emulsion structures, prolonging the stable time period of the emulsion, and preventing the emulsion composition from the decomposition for a long period of time. In addition, inositol is capable of thickening the components including collagen, guar gum, etc., and thus thickening of the components also enables the emulsion composition to be stabilized.

### [Glutathione]

Glutathione is a very simple molecule, and is constantly produced in the human body. Glutathione is a combined compound of glutamic acid, cysteine and glycine. Glutathione functions as an anti-oxidant in the living body, and is believed to combat free radicals which may damage cells in the human body. Glutathione imparts to the emulsion, an antioxidant characteristic for combating the free radicals which may destruct the emulsion. Glutathione plays an important role in many chemical reactions in the living body, and facilitates detoxication of chemical substances, pollutants and drugs.

Glutathione is an anti-oxidant, and is capable of eliminating oxygen. Thus resulting oxygen-scavenging property reduces the generation of bubbles, leading to an improvement of the emulsion stability of the emulsion composition. Furthermore, glutathione is capable of miniaturizing the emulsion structures, prolonging the stable time period of the emulsion, and preventing the emulsion composition from the decomposition for a long period of time.

### [Potassium Sorbate]

Potassium sorbate suppresses microorganisms such as molds and yeast in the emulsion composition. By using potassium sorbate as the enhancer, it is possible to suppress microorganisms derived from collagen, guar gum and the like. Since potassium sorbate is very highly soluble in aqueous solutions, superior effects of suppressing microorganisms in the emulsion composition are exhibited.

(Solvent)

The emulsion composition may contain any dispersion medium, and water and the like is exemplified.

(Other Components)

In addition to the components described above, the emulsion composition may also contain within a range not leading to impairment of the effects of the present invention, for example, a preservative, a pH adjusting agent, a defoaming agent, a thickening polysaccharide, an oxidation inhibitor, a coloring agent, a flavor, and the like.

Examples of the preservative include grapefruit seed extract, sodium hydroxymethylglycinate, phenoxyethanol, Japanese honeysuckle extract, citric acid, rosemary oleoresin, vitamin E, tea tree, and the like.

Examples of the pH adjusting agent include sodium citrate, ascorbic acid, acetic acid, tartaric acid, malic acid, fumaric acid, lactic acid, and the like.

Examples of the defoaming agent include simethicone.

### <pH of Emulsion Composition>

The lower limit of the pH of the emulsion composition is not particularly limited, and preferably 4.5, more preferably 5.0, and even more preferably 5.5. On the other hand, the upper limit of the pH is preferably 7.0, more preferably 6.5, and even more preferably 6.2. When the pH is less than the lower limit, coaggregation and precipitation may occur in the emulsion. To the contrary, when the pH is greater than the upper limit, the emulsion may be destabilized. In these regards, when the pH falls within the above range, it is possible to improve the emulsion stability of the emulsion composition, and also to prevent irritation to the skin.

### <Viscosity of Emulsion Composition>

The lower limit of the viscosity of the emulsion composition is preferably 500 mPa·s, more preferably 700 mPa·s, and even more preferably 900 mPa·s. On the other hand, the upper limit of the viscosity is preferably 10,000 mPa·s, more preferably 4,000 mPa·s, and even more preferably 2,000 mPa·s. When the viscosity falls within the above range, the emulsion stability of the emulsion composition can be improved. The “viscosity” as referred to herein means a viscosity determined using a type B viscometer, under a condition involving a temperature of 20° C. and a rotation speed of 30 rpm.

### <Production Method of Emulsion Composition>

The production method of an emulsion composition includes the mixing step of mixing the coconut oil, the lecithin and the co-emulsifier. By thus including the mixing step of mixing the coconut oil, the lecithin and the co-emulsifier, the production method of an emulsion composition enables an emulsion composition that is superior in emulsion stability to be easily and certainly produced. With the emulsion composition may be further mixed the enhancer described above. When the enhancer is further mixed with the emulsion composition, the emulsion composition that is more superior in the emulsion stability and has various types of functions can be produced. The production method of an emulsion composition may include a freeze-drying step of the mixture obtained by the mixing step.

The lower limit of the stirring time period in the mixing step is preferably 1 min, more preferably 2 min, and even more preferably 3 min. On the other hand, the upper limit of the stirring time period is preferably 10 min, more preferably 9 min, and even more preferably 8 min. When the stirring time period falls within the above range, the degree of emulsification of the emulsion composition can be improved, and thus the amount of the coconut oil absorbed into the living body can be increased.

The lower limit of the solution temperature in the mixing step is preferably 10° C., more preferably 15° C., and even more preferably 20° C. On the other hand, the upper limit of the solution temperature is preferably 40° C., more preferably 35° C., and even more preferably 30° C. When the solution temperature is higher than the upper limit, hydrophile-lipophile balance, i.e., HLB, may be impaired. To the contrary, when the solution temperature falls within the above range, the degree of emulsification of the emulsion composition can be improved, and the amount of the coconut oil absorbed into the living body can be increased.

The lower limit of the tip speed of the agitation blade of the mixer in the mixing step is preferably 800 m/min, more preferably 850 m/min, and even more preferably 900 m/min. On the other hand, the upper limit of the tip speed is preferably 1,300 m/min. more preferably 1,250 m/min, and even more preferably 1,200 m/min. When the tip speed falls within the above range, the micellar structures can be miniaturized, and the stability of the emulsion can be improved.

The procedure for freeze-drying of the emulsion composition is not particularly limited, and a well-known technique may be adopted. By freeze-drying the emulsion composition, the powdery matter can be obtained.

The emulsion composition according to the first embodiment produced by the production method described above contains a larger amount of the source of nutrients which had been contained in the coconut oil. More specifically, since an esterification reaction or the like for forming a glycerin fatty acid ester is not needed when the emulsion composition is produced, esterification of the source of nutrients during such an esterification reaction can be precluded. Therefore, it is possible to use the emulsion composition for, e.g., foods, beverages, medical drugs and the like.

### <Advantages>

Due to containing the coconut oil and the lecithin that is capable of forming a strong binding to the solvent, the emulsion composition can form micellar structures by way of the lecithin, whereby the emulsion stability can be improved. In addition, due to containing the co-emulsifier that strengthens the micellar structures, the emulsion composition enables the emulsion stability to be further improved.

### Second Embodiment

The emulsion composition according to the second embodiment contains a coconut oil, lecithin, a co-emulsifier, and deionized water. The second embodiment is different from the first embodiment in terms of further containing deionized water. When the emulsion composition thus further contains deionized water, fermentation of the emulsion composition is enabled.

### <Production Method of Emulsion Composition>

The production method of an emulsion composition includes: the mixing step of mixing the coconut oil, the lecithin, the co-emulsifier and deionized water; and a fermentation step of permitting fermentation of a mixture obtained in the mixing step. The production method of an emulsion composition may also include a step of freeze-drying an aqueous solution obtained in the fermentation step. Hereinafter, the description regarding the step and the like similar to those in the first embodiment will be omitted.

The microorganism which may be used in the fermentation step is exemplified by lactic acid bacteria, yeast, acetic acid bacteria and the like. Of these, lactic acid bacteria and yeast are preferred, and lactic acid bacteria are particularly preferred. It is possible to obtain a high fermentation rate by using the lactic acid bacteria as the microorganism for use in the fermentation of the mixture.

Examples of the lactic acid bacteria include those belonging to genus Lactobacillus such as Lactobacillus plantarum and Lactobacillus bulgaricus. Examples of the yeast include Saccharomyces cerevisiae, Candida utilis and the like. Furthermore, other microorganism which may be used in the fermentation step include Candida rugosa, Aspergillus oryzae, salmonella, Pseudomonas fluorescens, Escherichia coli, Bacillus substilis, and the like.

The lower limit of the fermentation time period in the fermentation step is preferably 24 hrs, more preferably 28 hrs, and even more preferably 32 hrs. On the other hand, the upper limit of the fermentation time period is preferably 48 hrs, more preferably 44 hrs, and even more preferably 40 hrs. When the fermentation time period is shorter than the lower limit, the fermentation may not be sufficiently proceed. On the other hand, when the fermentation time period is longer than the upper limit, a sour taste may be produced from the coconut oil.

The lower limit of the fermentation temperature in the fermentation step is preferably 10° C., more preferably 15° C., and even more preferably 20° C. On the other hand, the upper limit of the fermentation temperature is preferably 40° C., more preferably 35° C. and still more preferably 30° C. When the fermentation temperature is lower then the lower limit, the enzyme may be deactivated. To the contrary, when the fermentation temperature is higher than the upper limit, degradation of the components such as proteins and collagen may occur.

The fermentation brings about separation into an oily layer as the upper layer, and an aqueous layer as the lower layer. The aqueous solution as the lower layer is collected, with which a fermentation terminator is mixed to stop the fermentation process.

Examples of the fermentation terminator include ethanol, glucose, fructose, potassium sorbate and the like. Of these, ethanol and glucose are preferred, and ethanol is particularly preferred. When ethanol and/or glucose are/is selected as the fermentation terminator, influences on the human body can be decreased.

The emulsion composition according to the second embodiment does not contain a saccharide and thus is less sticky. Therefore, this emulsion composition can be used for, e.g., cosmetics and the like. In addition, the emulsion composition according to the second embodiment contain a larger amount of the source of nutrients which had been contained in the coconut oil, similarly to the emulsion composition according to the first embodiment.

### Other Embodiments

It should be construed that the embodiments disclosed herein are illustrative only and not in any how limiting the invention. The scope of the present invention is not restricted to the above constitutions of the embodiments, and it is intended to include the scope of the appended claims, including equivalent meanings and all modifications within the scope.

### EXAMPLES

Hereinafter, the present invention will be more specifically described by way of Examples, but the present invention is not limited to the following Examples.

### Example 1

A mixed solution was produced by mixing 25% by mass of a virgin coconut oil and 75% by mass of glycerol. To 1,000 g of the mixed solution was added 12 g of lecithin, and the mixture was stirred at 25° C. for 40 min at 600 rpm. When the average diameter of the droplets was 2.0 rpm, the stirring was stopped to obtain an emulsion composition of Example 1.

### Example 2

A mixed powder was prepared by adding 50 g of inositol, 50 g of glutathione and 50 g of collagen to 12 g of lecithin. In addition, a mixed solution was produced by mixing 25% by mass of a virgin coconut oil and 75% by mass of glycerol. To 1,000 g of the mixed solution was added 50 g of the mixed powder described above, and the mixture was stirred at 25° C. for 40 min at 600 rpm. When the average diameter of the droplets was 1.0 μm, the stirring was stopped to obtain an emulsion composition of Example 2.

### Example 3

A second mixed solution was prepared by mixing 300 mL of virgin coconut oil and 700 mL deionized water. To 1,000 g of the second mixed solution was added 50 g of the mixed powder of Example 2, and the mixture was stirred at 25° C. for 40 min at 600 rpm. When the average diameter of the droplets was 1.0 μm, the stirring was stopped to obtain a mixture. Thereafter, the mixture was subjected to fermentation for 24 hrs. The fermentation brought about separation into an oily layer as the upper layer, and an aqueous layer as the lower layer, and 500 mL of the aqueous solution as the lower layer was collected, which was mixed with 50 mL of ethanol to stop the fermentation reaction. Accordingly, an emulsion composition of Example 3 was obtained.

### [Evaluation]

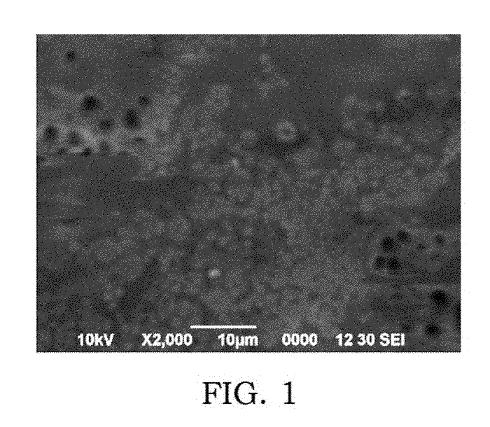

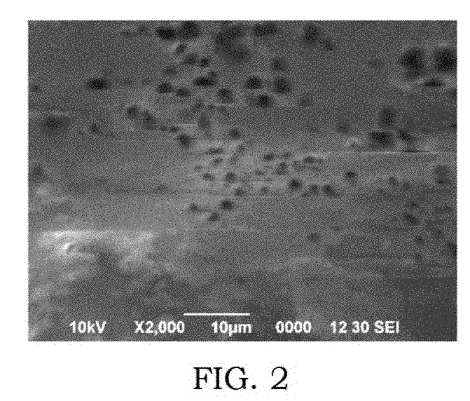

FIG. 1 shows an SEM image of the emulsion composition of Example 1, taken by a scanning electron microscope (hereinafter, may be also referred to as “SEM”), and FIG. 2 shows an SEM image of the emulsion composition of Example 2. As shown in FIG. 1, it was confirmed that a stable emulsion was formed when a solution containing the virgin coconut oil, the lecithin and glycerol was mixed by stirring. Furthermore, as shown in FIG. 2, it was confirmed that when an enhancer was further mixed therewith in addition to the lecithin, finer emulsion structures were formed accompanied by less generation of bubbles, and thus a more stable emulsion was formed. Consequently, it was ascertained that the emulsion composition according to the present invention was able to form a highly stable emulsion.

### INDUSTRIAL APPLICABILITY

As described in the foregoing, the emulsion composition according to the present invention can be suitably used for health supplements, aromatherapies, foods, beverages, medical drugs and cosmetics, owing to superior emulsion stability.

What is claimed is:

1. An emulsion composition comprising:
a coconut oil;
a lecithin; and
a co-emulsifier.

2. The emulsion composition according to claim 1, wherein the co-emulsifier is a polyhydric alcohol having at least two hydroxyl groups.

3. The emulsion composition according to claim 2, wherein the polyhydric alcohol is glycerol.

4. The emulsion composition according to claim 3, further comprising glucose, galactose, mannose, lactose, fructose, maltose, sucrose, xylose, trehalose, propylene glycol, sorbitol, xylitol, mannitol, erythritol, threitol, arabitol, dulcitol, iditol, ribitol, lactitol, maltitol or isomalt as the co-emulsifier.

5. The emulsion composition according to claim 1, further comprising deionized water.

6. The emulsion composition according to claim 1, further comprising honey, collagen, inositol, glutathione or potassium sorbate.

7. A powdery matter comprising:
a coconut oil;
a lecithin; and
a co-emulsifier.

8. The powdery matter according to claim 7 which is water soluble.

9. A production method of an emulsion composition comprising mixing a coconut oil, a lecithin and a co-emulsifier.
